(19) United States
(12) Patent Application Publication
(10) Pub. No.: US 2019/0083670 A1
(43) Pub. Date: Mar. 21, 2019

(54) AROMA DIFFUSER

(71) Applicant: Audiowell Electronics (Guangdong) Co., Ltd., GuangZhou (CN)

(72) Inventors: Zhenjie Zheng, Guangzhou (CN); Caiyuan Yang, Guangzhou (CN); Hao Fu, Guangzhou (CN)

(73) Assignee: Audiowell Electronics (Guangdong) Co., Ltd., Guangzhou (CN)

(21) Appl. No.: 15/567,277

(22) PCT Filed: Jun. 7, 2017
(86) PCT No.: PCT/CN2017/087385

§ 371 Date: Oct. 17, 2017

(30) Foreign Application Priority Data
Jun. 7, 2016 (CN) 201610402129.4

(51) Int. Cl. A61L 9/12

(52) U.S. Cl. CPC A61L 9/122

(57) ABSTRACT

An aroma diffuser includes a liquid storage means, an enclosure provided with a mounting cavity, a gas supply means and a seal. The enclosure is covered on a liquid storage mean. The gas supply means and the liquid storage means are both aranged in the mounting cavity and communicate with each other. The gas supply means, the liquid storage means and the enclosure cooperate to form a gas return passage. The seal is arranged between the liquid storage means and the gas supply means, located in the gas return passage and sealingly abuts on the enclosure. The gas return passage can be blocked by the seal, aroma gas produced by the aroma diffuser is prevented from flowing back from the gas return passage into the gas supply means to reduce the concentration of the aroma gas released into the environment and thus to affect the use effect of the aroma diffuser.

### FIELD

The present disclosure relates to the field of gas diffusion device, and more particularly, to an aroma diffuser.

### BACKGROUND

An aroma diffuser is a device that transforms aroma liquid or essential oil from a liquid form to an aerosol form through heating or ultrasonic oscillation, and delivers the aerosol to ambient air with a high-pressure gas pump, to provide for breathing. It can achieve health-care effects such as accelerating metabolism, promoting cells regeneration, enhancing immunity and so on. Currently available aroma diffusers may generally include a configuration made up of a pump and a specific nozzle, in which the pump may generate a high-speed gas flow and creates a localized low-pressure region to form a difference in pressure, and the aerosol essential oil may be brought to the nozzle with the gas flow and enter the external environment through a gas outlet of the aroma diffuser, so that the function of aroma diffusing is achieved. In the aroma diffuser employing the above working principle, an outlet of an aroma liquid container and an inlet of the pump communicate with each other, which causes that the aroma gas would be sucked into the pump when it spreads into the pump mounting area, thereby greatly reducing the concentration of the aroma gas ejected from the outlet of the aroma diffuser and affecting the working effect of the aroma diffuser. Furthermore, since the aroma liquid is acidic, when the aerosol aroma liquid (essential oil or perfume) enters the pump, or the aroma liquid directly contacts the pump as the aroma liquid container is tilted or turned upside down, elements of the pump would be corroded and cannot work normally, thereby greatly affecting the service life of the aroma diffuser.

### SUMMARY

In view of the above, in order to overcome the defects in the prior art, the present disclosure provides an aroma diffuser, which can prevent backflow of aroma gas and avoid corrosion and damage, and has a simple structure, improved operation reliability and service life.

An objective of the disclosure is achieved by the following solution.

An aroma diffuser includes a liquid storage means, an enclosure provided with a mounting cavity, a gas supply means and a seal, the enclosure is covered on the liquid storage means, the gas supply means and the liquid storage means are both arranged in the mounting cavity and communicate with each other, the gas supply means, the liquid storage means and the enclosure cooperate to form a gas return passage, and the seal is arranged between the liquid storage means and the gas supply means, located in the gas return passage and sealingly abuts on the enclosure.

Further details of the above solution are described as below.

Further, the enclosure is provided thereon with a first gas intake passage and a first gas exit passage, the gas supply means includes a gas driving member having a pressurized gas passage, the liquid storage means includes a rotary cover, a gas supply passage is formed between the seal and the rotary cover, the rotary cover is provided with a second gas intake passage and a second gas exit passage, the second gas intake passage communicates with the pressurized gas passage, and the second gas exit passage sequentially communicates with the gas supply passage and the first gas exit passage; and

wherein the first gas intake passage, the gas supply passage and the first gas exit passage are arranged parallel to each other, the pressurized gas passage, the second gas intake passage and the second gas exit passage are arranged parallel to each other.

Further, the seal is rotatably fixed to the rotary cover, and the first gas exit passage includes a first gas outlet mated with the gas supply passage.

Further, the aroma diffuser further includes a positioning member, the rotary cover includes a connecting post, the pressurized gas passage includes a pressurized gas outlet, the second gas intake passage includes a second gas inlet, the positioning member is sleeved on the connecting post and positioned between the gas driving member and the seal, and the pressurized gas outlet mates with and abuts against the second gas inlet.

Further, the positioning member is provided with a groove, and the connecting post is embedded in the groove.

Further, the positioning member is provided with a fitting hole, the gas driving member is provided with an engaging stud, and the engaging stud is engaged in the fitting hole.

Further, the gas supply means further includes a control element electrically connected to the gas driving member, the seal is provided thereon with a supporting mechanism, and the control element is arranged on the supporting mechanism.

Further, the liquid storage means further includes a gas delivery conduit, and a liquid storage bottle connected with the rotary cover, one end of the gas delivery conduit is connected with the second gas intake passage, and another end of the gas delivery conduit extends below liquid level of the liquid storage bottle.

Further, an inner wall the rotary cover is provided with a first thread structure, an outer wall of a bottle mouth of the liquid storage bottle is provided with a second thread structure mated with the first thread structure, and the liquid storage bottle mates with the rotary cover.

The present disclosure has the following beneficial effects.

By arranging the seal between the liquid storage means and the gas supply means and ensuring that the seal is located in the gas return passage to form a seal connection structure with the enclosure, the gas return passage can be blocked by the seal. In this way, aroma gas produced by the aroma diffuser is prevented from flowing back from the gas return passage into the gas supply means to reduce the concentration of the aroma gas released into the environment, and thus to affect the use effect of the aroma diffuser. Meanwhile, the acidic aroma gas is prevented from entering the gas supply means to corrode the elements disposed therein and thus to reduce operation reliability and service life of the aroma diffuser.

### BRIEF DESCRIPTION OF DRAWINGS

FIG. 1 is an exploded diagram illustrating an aroma diffuser according to an embodiment of the present disclosure.

FIG. 2 is a structural schematic diagram illustrating an aroma diffuser according to an embodiment of the present disclosure.

FIG. 3 is a structural schematic diagram illustrating a seal of an aroma diffuser according to an embodiment of the present disclosure;

FIG. 4 is a structural schematic diagram illustrating a rotary cover of an aroma diffuser according to an embodiment of the present disclosure.

### DESCRIPTION OF REFERENCE SIGNS

100: liquid storage means; 120: rotary cover; 122: second gas intake passage; 123: second gas inlet; 124: second gas exit passage; 125: connecting post; 140: gas delivery conduit; 160: liquid storage bottle; 162: first thread structure; 164: second thread structure; 200: enclosure; 220: mounting cavity; 240: first gas intake passage; 260: first gas exit passage; 262: first gas inlet; 300: gas supply means; 320: gas driving member; 322: pressurized gas passage; 324: pressurized gas outlet; 326: engaging stud; 340: control element; 400: seal; 420: supporting mechanism; 500: gas return passage; 600: gas supply passage; 700: positioning member; 720: groove; and 740: fitting hole.

### DETAILED DESCRIPTION OF PREFERRED EMBODIMENTS

The present disclosure will be further described in detail as follows.

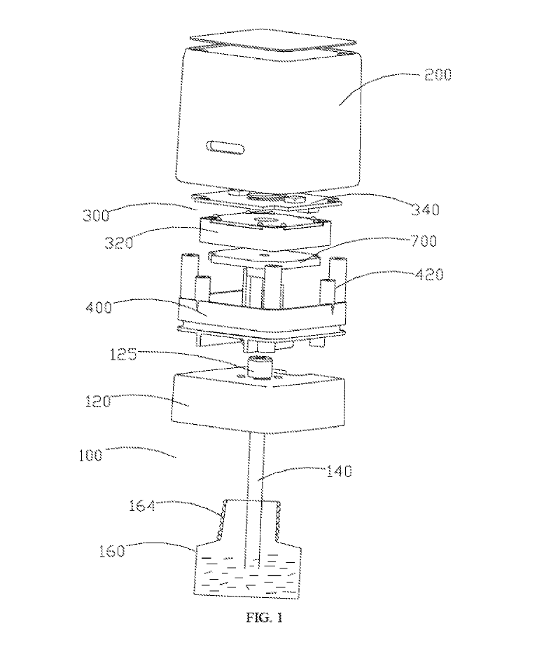

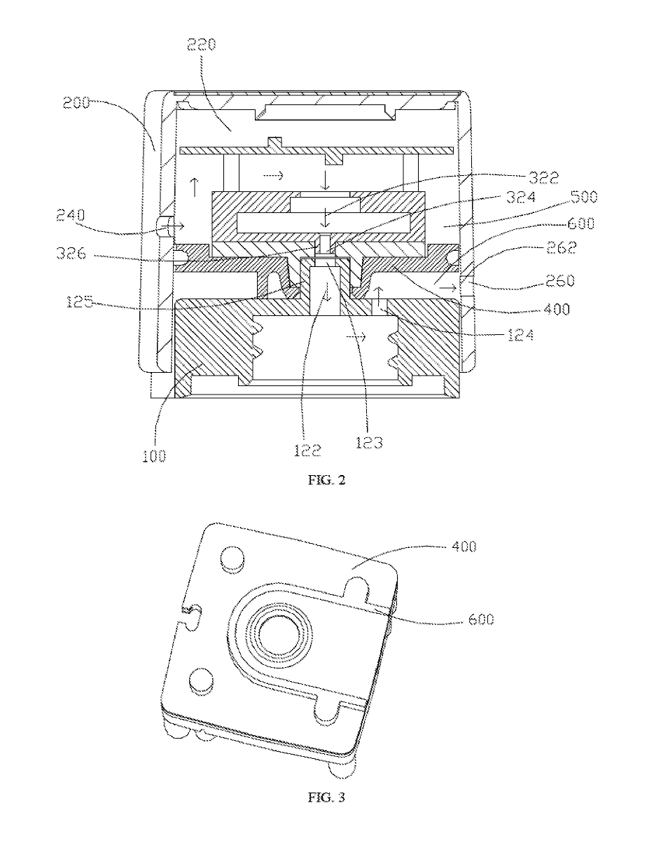

As illustrated in FIG. 1 and FIG. 2, an aroma diffuser may include a liquid storage means 100, an enclosure 200 provided with a mounting cavity 220, a gas supply means 300 and a seal 400. The enclosure 200 may be covered on the liquid storage means 100. The gas supply means 300 and the liquid storage means 100 may be both provided in the mounting cavity 220 and communicate with each other. The gas supply means 300, the liquid storage means 100 and the enclosure 200 may cooperate to form a gas return passage 500. The seal 400 may be arranged between the liquid storage means 100 and the gas supply means 300, located in the gas return passage 500 and sealingly abuts on the enclosure 200.

By arranging the seal 400 between the liquid storage means 100 and the gas supply means 300 and ensuring that the seal 400 is located in the gas return passage 500 to form a seal connection structure with the enclosure 200, the seal 400 can block the gas return passage 500. In this way, aroma gas produced by the aroma diffuser is prevented from flowing back from the gas return passage 500 into the gas supply means 300 to reduce the concentration of aroma gas released into the environment and thus to affect the use effect of the aroma diffuser, and meanwhile, the acidic aroma gas is prevented from entering the gas supply means 300 to corrode the elements disposed therein and thus to reduce operation reliability and service life of the aroma diffuser. And when the enclosure 200 and the gas storage means are mated and connected with each other, the connection has good sealing performance so as to prevent the produced aroma gas from leakage. In addition, in this embodiment, the gas return passage 500 is a part of the mounting cavity 220, thus there is no need to design a special structure to form a gas return passage, which would increase the product design and manufacture costs. Sealed abutment of the seal 400 on the enclosure 200 specifically means that the outer circumferential wall of the seal 400 is in close contact with the inner wall of the enclosure 200 so as to obtain sealing effect and prevent aroma gas from passing through.

Moreover, the gas supply means 300 may further include a control element 340 electrically connected to the gas driving member 320. The seal 400 may be provided thereon with a supporting mechanism 420. The control element 340 may be arranged on the supporting mechanism 420. In this embodiment, the control element 340 may be a printed circuit board (PCB) welded thereon with a control chip. The control chip may be operable via an operation panel disposed externally on the enclosure 200 to turn on or off of a piezoelectric pump. The seal 400 may be provided thereon with at least two posts, i.e. the supporting mechanism 420. By arranging the PCB on the supporting mechanism 420, good supporting effect can be achieved. Further, secure fitting can be realized by using locking elements, such as screws, thereby making that the aroma diffuser has a simple and compact structure and thus has a reduced whole volume.

Referring to FIG. 2, the enclosure 200 may be further provided thereon with a first gas intake passage 240 and a first gas exit passage 260. The gas supply means 300 may include a gas driving member 320 having a pressurized gas passage 322. The liquid storage means 100 may include a rotary cover 120. A gas supply passage 600 may be formed between the seal 400 and the rotary cover 120. The rotary cover 120 may be provided with a second gas intake passage 122 and a second gas exit passage 124. The second gas intake passage 122 may communicate with the pressurized gas passage 322, and the second gas exit passage 124 may sequentially communicate with the gas supply passage 600 and the first gas exit passage 260. The first gas intake passage 240, the gas supply passage 600 and the first gas exit passage 260 may be arranged parallel to each other, and the pressurized gas passage 322, the second gas intake passage 122 and the second gas exit passage 124 may be arranged parallel to each other.

In this embodiment, by arranging the first gas intake passage 240, the gas supply passage 600 and the first gas exit passage 260 parallel to each other, and arranging the pressurized gas passage 322, the second gas intake passage 122 and the second gas exit passage 124 parallel to each other, a meander-shaped gas passage can be formed inside the present aroma diffuser, and thus backflow of gas inside the aroma diffuser, which would affect normal operation and use effect of the aroma diffuser, can be avoided.

The first gas intake passage 240 and the first gas exit passage 260 are preferably defined as through holes provided in the opposite surfaces of the enclosure 200. The first gas intake passage 240 may be configured to introduce air into the aroma diffuser, and the first gas exit passage 260 may be configured to discharge the aroma gas produced by the aroma diffuser into the environment for people to use. In addition, the gas driving member 320 is preferably a piezoelectric pump, and provided with a pressurized gas passage 322 passing therethrough. The piezoelectric pump may be designed to be pressurized by the piezoelectric pump so that air flow from the first gas intake passage 240 may be pressurized to a certain pressure, and may be also designed to ensure that the pressurized gas passage 322 communicates with the second gas intake passage 122 after the air flow has been pressurized to the certain pressure. In addition, the second gas exit passage 124 may be defined as a through hole provided in a top surface of the rotary cover 120 and close to the first gas intake passage 240, which is sequentially communicate with the gas supply passage 600 and the first gas exit passage 260, so as to transform the pressurized gas into aerosol aroma gas and discharge the aerosol aroma gas from the aroma diffuser. Herein, the gas supply passage 600 with a certain length has cushioning effect on the pressurized aroma gas flowing at high speed, so as to prevent generation of noise and vibration of the aroma diffuser to affect user experience when the aroma gas is directly discharged from the first gas exit passage 260.

As illustrated in FIG. 2 and FIG. 3, the seal 400 may be rotatably fixed to the rotary cover 120. The first gas exit passage 260 may include a first gas inlet 262 mated with the gas supply passage 600. In this embodiment, the seal 400 is a block, both sides of which closely abuts against the inside wall of the enclosure 200, thus the seal 400 can rotate with the enclosure 200. In addition, the block is provided therein with a U-shaped chamber including the gas supply passage 600, and the gas outlet of the gas supply passage 600 is connected with the first gas exit passage 260. The first gas exit passage 260 is preferably an elongated channel-shaped opening with a first length, therefore, by rotating the enclosure 200 to rotate the U-shaped chamber, the U-shaped chamber can be engaged with the elongated channel-shaped opening in various directions and the direction of discharging the aroma gas from the first gas exit passage 260 can be changed, thereby flexibly controlling the gas discharging direction of the aroma diffuser, realizing directional injection, supplying more aroma gas to target regions, and improving the flexibility and experience of use.

Based on the above embodiments, the above aroma diffuser may further include a positioning member 700. The rotary cover 120 may include a connecting post 125. The pressurized gas passage 322 may include a pressurized gas outlet 324. The second gas intake passage 122 may include a second gas inlet. The positioning member 700 may be sleeved on the connecting post 125 and positioned between the gas driving member 320 and the seal 400. The pressurized gas outlet 324 may mate with and abut against the second gas inlet 123.

In this embodiment, the positioning member 700 is preferably a pad made of rubber, and the pad is closely engaged in a slot of the seal 400 having a shape fitting the shape of the pad, thus the pad can rotate with the seal 400. Then the piezoelectric pump may be arranged on the pad, which can avoid hard-to-hard direct press of the piezoelectric pump and the seal 400 to cause damage. In addition, the auxiliary positioning effect of the pad can ensure that the pressurized gas outlet 324 and the second gas inlet 123 correctly abut against each other, i.e., centers of the two cylindrical through holes coincide with each other, so as to ensure smooth delivery of gas.

Further, the positioning member 700 may be provided with a groove 720. The connecting post 125 may be embedded in the groove 720. The connecting post 125 may mate and engage with the groove 720, making the assembled structure more compact and the connection more securely, therefore, when the rotary cover 120 rotates relative to the seal 400, the seal 400 is prevented from moving laterally to make poor alignment of the pressurized gas outlet 324 and the second gas inlet 123, affecting delivery of the pressurized gas, making gas supply of the aroma diffuser insufficient and affecting normal operation of the aroma diffuser.

In addition, the positioning member 700 may further include a fitting hole 740. The gas driving member 320 is provided with an engaging stud engaged in the fitting hole 740. In this embodiment, the fitting hole 740 may be recessed inwardly from the top surface of the pad, and mated and engaged with the engaging stud projecting on the piezoelectric pump, thereby ensuring secure connection between the pad and the piezoelectric pump and making structure more compact.

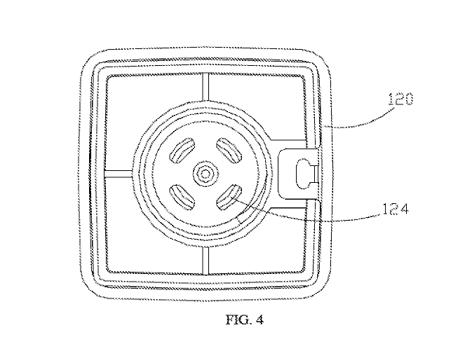

Referring to FIG. 2 and FIG. 4, the liquid storage means 100 may further include a gas delivery conduit 140 and a liquid storage bottle 160 connected with the rotary cover 120. One end of the gas delivery conduit 140 may be connected with the second gas intake passage 122, and another end of the gas delivery conduit 140 may extend below liquid level of the liquid storage bottle 160. In this embodiment, pressurized gas produced by the piezoelectric pump may pass through the gas delivery conduit 140 and enter the liquid storage bottle 160 to make the aroma liquid (perfume or essential oil) boiling, accelerating evaporation of the aroma liquid and transforming the aroma liquid into aerosol aroma gas. Compared with traditional aroma diffusers which operate with a blower or by ultrasonic concussion, the above technical solutions have simpler working principle and better transforming effect and avoid generation of noise and vibration which affect experience in use.

Further, an inner wall of the rotary cover 120 is provided with a first thread structure 162, and an outer wall of a bottle mouth of the liquid storage bottle 160 is provided with a second thread structure 164 mated with the first thread structure 162. The liquid storage bottle 160 may mate with the rotary cover 120. The thread connection structure can facilitate assembly, disassembly and replacement of the liquid storage bottle 160, thereby improving convenience and experience in use of the aroma diffuser.

The technical features of the above-described embodiments can be arbitrarily combined. For simplicity, not all possible combinations of the technical features in the above embodiments are described. However, the combinations shall fall into the scope of the present disclosure as long as there is no contradiction among the combinations of these technical features.

What described above are several embodiments of the present disclosure, and they are specific and in details, but not intended to limit the scope of the present disclosure. It will be understood by those skilled in the art that various modifications and improvements can be made without departing from the concept of the present disclosure, and all these modifications and improvements are within the scope of the present disclosure. The scope of the present disclosure shall be subject to the claims attached.

What is claimed is:

1. An aroma diffuser, comprising:
a liquid storage means;
an enclosure provided with a mounting cavity; and
a gas supply means and a seal,
wherein the enclosure is covered on a liquid storage means, the gas supply means and the liquid storage means are both arranged in the mounting cavity and communicate with each other, the gas supply means, the liquid storage means and the enclosure cooperate to form a gas return passage, and the seal is arranged between the liquid storage means and the gas supply means, located in the gas return passage and sealingly abuts on the enclosure.

2. The aroma diffuser of claim 1, wherein the enclosure is provided thereon with a first gas intake passage and a first gas exit passage, the gas supply means includes a gas driving member having a pressurized gas passage, the liquid storage means includes a rotary cover, a gas supply passage is formed between the seal and the rotary cover, the rotary cover is provided with a second gas intake passage and a second gas exit passage, the second gas intake passage communicates with the pressurized gas passage, and the second gas exit passage sequentially communicates with the gas supply passage and the first gas exit passage; and
wherein the first gas intake passage, the gas supply passage and the first gas exit passage are arranged parallel to each other, and the pressurized gas passage, the second gas intake passage and the second gas exit passage are arranged parallel to each other.

3. The aroma diffuser of claim 2, wherein the seal is rotatably fixed to the rotary cover, and the first gas exit passage includes a first gas outlet mated with the gas supply passage.

4. The aroma diffuser of claim 2, further comprising a positioning member,
wherein the rotary cover includes a connecting post, the pressurized gas passage includes a pressurized gas outlet, the second gas intake passage includes a second gas inlet, the positioning member is sleeved on the connecting post and positioned between the gas driving member and the seal, and the pressurized gas outlet mates with and abuts against the second gas inlet.

5. The aroma diffuser of claim 4, wherein the positioning member is provided with a groove, and the connecting post is embedded in the groove.

6. The aroma diffuser of claim 4, wherein the positioning member is provided with a fitting hole, the gas driving member is provided with an engaging stud, and the engaging stud is embedded in the fitting hole.

7. The aroma diffuser of claim 2, wherein the gas supply means further includes a control element electrically connected to the gas driving member, the seal is provided thereon with a supporting mechanism, and the control element is arranged on the supporting mechanism.

8. The aroma diffuser of claim 2, wherein the liquid storage means further includes a gas delivery conduit and a liquid storage bottle connected with the rotary cover, one end of the gas delivery conduit is connected with the second gas intake passage, and another end of the gas delivery conduit extends below liquid level of the liquid storage bottle.

9. The aroma diffuser of claim 8, wherein an inside wall of the rotary cover is provided with a first thread structure, an outer wall of a bottle mouth of the liquid storage bottle is provided with a second thread structure mated with the first thread structure, and the liquid storage bottle mates with the rotary cover.
